(19) United States
(12) Patent Application Publication
(10) Pub. No.: US 2008/0124671 A1
(43) Pub. Date: May 29, 2008

(54) In situ powered hand dental instrument

(76) Inventors: Bao Luu Nguyen, Diamond Bar, CA (US); Liet Hong Nguyen, Garden Grove, CA (US)

Correspondence Address: BLAKELY SOKOLOFF TAYLOR & ZAFMAN, 1279 OAKMEAD PARKWAY, SUNNYVALE, CA, 94085-4040, US

(21) Appl. No.: 11/985,165

(22) Filed: Nov. 13, 2007

Related U.S. Application Data
(60) Provisional application No. 60/858,745, filed on Nov. 13, 2006.

(51) Int. Cl. A61C 3/00; A61B 1/247

(52) U.S. Cl. 433/30; 433/32; 433/140; 433/141

(57) ABSTRACT

A multi-functional dental instrument has a base handle and a first attachment. The base handle has first and second base ends opposite to each other. The base handle comprises a power connector to connect to a power source and a first base connector at the first base end. The first attachment is coupled to the first base connector at the first base end. The first attachment is powered by the power source to perform a first dental task at a working area.

Another embodiment includes a bite block, a holder embedded in the bite block, and a light source attached to the holder to illuminate an operation field. The light source is powered by a power source.

Another embodiment includes an absorbent pad and an illuminator having a light source attached to the absorbent pad to generate light when powered by a power source.

### RELATED APPLICATIONS

This patent application claims the benefits of (1) US Provisional Application, titled “In situ powered hand instrument and composite light cure”, Ser. No. 60/858,745, filing date: Nov. 13, 2006.

### BACKGROUND

1. Field of the Invention

Embodiments of the invention relate to the field of dentistry, and more specifically, to dental instrument.

2. Description of Related Art

Dental instruments are used by dentists in working on patient's teeth. Due to the delicate nature of dental work, these instruments are made to provide ease of use, space efficiency, and convenience. Examples of these instruments include dental scalers, probes and explorers, excavators, spatulas, mouth mirrors, etc.

Existing techniques for dental instruments have a number of disadvantages. First, a majority of these instruments are intended for one specific use. Each instrument may only perform one specific task. For example, a carver is used to carve anatomy on silver fillings; a condenser is used to press the silver filling into place, an amalgam carrier scoops up the amalgam (silver filling material) and places into the opening of the tooth, etc. When a dentist is working on a patient's mouth, he or she has to change instruments as often as necessary to complete a procedure. Since a dental procedure typically involves a number of tasks, a dentist has to change several instruments several times to perform the procedure. This causes inefficiency and inconvenience to the dentist and also causes discomfort to the patient. Second, many of these instruments are intended for manual use, not for powered use. Some dental task may be facilitated by operating with power to reduce fatigue and stress to the dentist. Third, some instruments are bulky. For example, light curing instruments using halogen bulb or light-emitting diode (LED) are typically large and cumbersome.

### BRIEF DESCRIPTION OF THE DRAWINGS

Embodiments of invention may best be understood by referring to the following description and accompanying drawings that are used to illustrate embodiments of the invention. In the drawings:

FIG. 1 is a diagram illustrating a system in which one embodiment of the invention can be practiced.

FIG. 2A is a diagram illustrating a first attachment as a curing unit with one light source according to one embodiment of the invention.

FIG. 2B is a diagram illustrating a first attachment as a curing unit with two light sources according to one embodiment of the invention.

FIG. 2C is a diagram illustrating a first attachment as a curing unit with two light sources and an optical filter according to one embodiment of the invention.

FIG. 3A is a diagram illustrating a second attachment as a hollow enclosure according to one embodiment of the invention.

FIG. 3B is a diagram illustrating a second attachment as an illuminator according to one embodiment of the invention.

FIG. 3C is a diagram illustrating a second attachment as a heating unit according to one embodiment of the invention.

FIG. 3D is a diagram illustrating a second attachment as a vibrator unit according to one embodiment of the invention.

FIG. 3E is a diagram illustrating a second attachment as an illuminator and vibrator unit according to one embodiment of the invention.

FIG. 4A is a diagram illustrating an instrument tip as a spatula according to one embodiment of the invention.

FIG. 4B is a diagram illustrating an instrument tip as a mirror according to one embodiment of the invention.

FIG. 4C is a diagram illustrating an instrument tip as a transluminator according to one embodiment of the invention.

FIG. 4D is a diagram illustrating an instrument tip as a periodontal probe according to one embodiment of the invention.

FIG. 4E is a diagram illustrating an instrument tip as a cement spatula according to one embodiment of the invention.

FIG. 4F is a diagram illustrating an instrument tip as a endodontic explorer according to one embodiment of the invention.

FIG. 4G is a diagram illustrating an instrument tip as a bite bloc according to one embodiment of the invention.

FIG. 4H is a diagram illustrating an instrument tip as a gutta percha heater according to one embodiment of the invention.

FIG. 4I is a diagram illustrating an instrument tip as a wax spatula according to one embodiment of the invention.

FIG. 4J is a diagram illustrating an instrument tip as a heating tip according to one embodiment of the invention.

FIG. 5A is a diagram illustrating an attachment having an illuminating assembly attached to a suction tube according to one embodiment of the invention.

FIG. 5B is a diagram illustrating an attachment having an illuminating assembly attached to a mirror according to one embodiment of the invention.

FIG. 6 is a diagram illustrating an attachment having an imaging device according to one embodiment of the invention.

FIG. 7 is a flowchart illustrating a process to use the multi-functional dental instrument according to one embodiment of the invention.

FIG. 8A is a diagram illustrating a bite block assembly according to one embodiment of the invention.

FIG. 8B is a diagram illustrating a bite block assembly used in a patient's mouth according to one embodiment of the invention.

FIG. 9A is a diagram illustrating an absorbent pad assembly according to one embodiment of the invention.

FIG. 9B is a diagram illustrating an absorbent pad assembly used in a patient's mouth according to one embodiment of the invention.

FIG. 10A is a diagram illustrating an adjustable bite block assembly according to one embodiment of the invention.

FIG. 10B is a diagram illustrating an adjustable bite block assembly used in a patient's mouth according to one embodiment of the invention.

### DESCRIPTION

An embodiment of the present invention is a multi-functional dental instrument. A base handle has first and second base ends opposite to each other. The base handle comprises a power connector to connect to a power source, a first base connector at the first base end, and a second base connector at the second base end. A first attachment is coupled to the first base connector at the first base end. The first attachment is powered by the power source to perform a first dental task at a working area. An instrument tip is coupled directly or indirectly to the base handle at the second base end to perform a second dental task different than the first dental task. A second attachment has first and second attachment ends opposite to each other. The first attachment end is coupled to the second base connector at the second base end. The first or second attachment may be attached to the instrument tip to operate the instrument tip.

Another embodiment includes a bite block, a holder embedded in the bite block, and a light source attached to the holder to illuminate an operation field. The light source is powered by a power source.

Another embodiment includes an absorbent pad and an illuminator having a light source attached to the absorbent pad to generate light when powered by a power source.

In the following description, numerous specific details are set forth. However, it is understood that embodiments of the invention may be practiced without these specific details. In other instances, well-known circuits, structures, and techniques have not been shown to avoid obscuring the understanding of this description.

One embodiment of the invention may be described as a process which is usually depicted as a flowchart, a flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed. A process may correspond to a method, a program, a procedure, a method of manufacturing or fabrication, etc.

One embodiment of the invention is a in situ powered hand multi-functional dental instrument. The instrument is compact, versatile, and convenient to use. It provides the dentist and his or her dental assistant a versatile tool to perform dental procedures on a patient in a very efficient manner. Another embodiment includes a bite block, a holder embedded in the bite block, and a light source attached to the holder to illuminate an operation field. The light source is powered by a power source. Another embodiment includes an absorbent pad and an illuminator having a light source attached to the absorbent pad to generate light when powered by a power source.

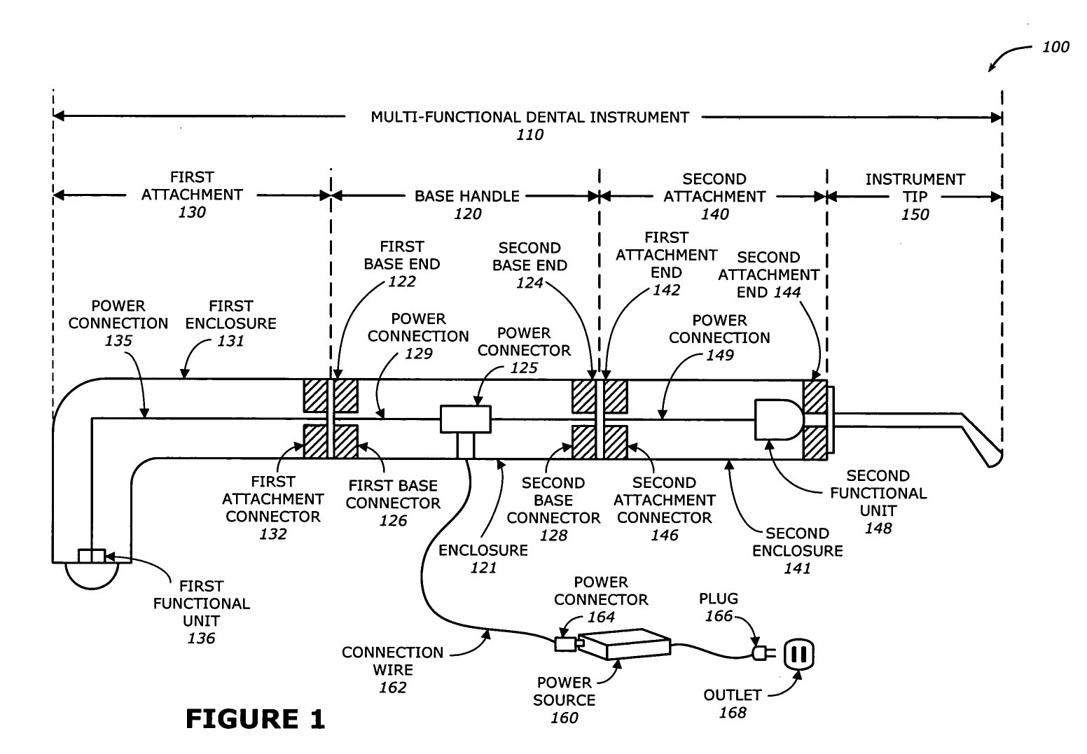

FIG. 1 is a diagram illustrating a system 100 in which one embodiment of the invention can be practiced. The system 100 includes a multi-functional dental instrument 110 and a power source 160. The dental instrument 110 may include a base handle 120, a first attachment 130, a second attachment 140, and an instrument tip 150. Note that the system 100 and/or the dental instrument 110 may include more or less than the above components. For example, the second attachment 140 and the instrument tip 150 may not be needed in some configurations.

The instrument 110 is connected to the power source 160 via a connection wire 162 and a power connector 164. The power source 160 may be any suitable power source that may generate direct current (DC) power to power the instrument 110. The power source 160 may be plugged into an electrical outlet 168 via a plug 166 to convert alternating current (AC) (e.g., 120 VAC to 220 VAC) to a suitable DC voltage (e.g., 9 VDC to 20 VDC). The power source 160 may also be a DC power source (e.g., batteries) located inside the base handle 120 if it is compact enough.

The base handle 120 has a first base end 122 and a second base end 124 opposite to each other. The length of the base handle 120 may be any suitable length. It may range from one inch to six inches. The base handle 120 may include a housing 121 that may be constructed by any suitable material (e.g., plastic, metal, metal alloy, or any combination of them). The housing 121 may be an elongated hollow structure having a cross section of any suitable shape such as circle, hexagonal, square, rectangle, etc. In other words, it may have a shape of a cylinder, a rectangular prism, a hexagonal prism, etc. The diameter of the base handle may be about 7 mm. The housing 121 may house or contain a power connector 125 to connect to the power source 160, a first base connector 126 at the first base end 122, and a second base connector 128 at the second base end 124. The power connector 125 may be located inside the base handle 120. It has power connection 129 to connect the first base connector 126 and the second base connector 128. The power connection 129 may be any suitable conductor to conduct electricity or carry power.

The first attachment 130 is attached to the base handle 120 at the first base end 122. It may be coupled to the first base connector 126 at the first base end 122. The first attachment 130 may be powered by the power source 160 to perform a first dental task at a working area. The first dental task may be any suitable task in a dental procedure (e.g., curing). The working area is typically the area inside the patient's mouth where dental work is being performed.

The first attachment 130 may include a first attachment connector 132, a first enclosure 131, and a first functional unit 136. The first attachment connector 132 may be mechanically compatible with the first base connector 126 such that the first attachment 130 is coupled to the first base connector 126 via a fast action (e.g., a snap action). The first enclosure 131 may be hollow and have a first length. Its shape may be any suitable shape and may be compatible with the base handle 120. For example, its diameter may be approximately 7 mm. The first functional unit 136 may be placed inside the first hollow enclosure 131 and operate by receiving power from the power source 160 via the power connector 125.

The instrument tip 150 is coupled to the base handle 120 directly or indirectly at the first base end 122 (via the first attachment 130) or the second base end 124 (via the second attachment 140) to perform a second dental task different than the first dental task. The instrument tip 150 may be attached directly to the base handle 120 at the second base end 124 via the second base connector 128. It may also be attached indirectly to the base handle 120 through the first attachment 130 or the second attachment 140. By having at least two elements to perform different dental tasks in one instrument, the dental instrument 110 provides convenience and efficiency to the dentist. The dentist may use the first attachment to perform the first task. Then, he or she may turn the instrument around to use the instrument tip 150 to perform the second task.

The second attachment 140 may have a first attachment end 142 and a second attachment end 144 opposite to each other. The first attachment end 142 may be coupled to the second base connector 128 of the base handle 120 at the second base end 124. It may be attached to the instrument tip 150 at the second attachment end 144 to operate the instrument tip 150. Similarly, the first attachment 130 may also be attached to the instrument tip 150 to operate the instrument tip 150.

The second attachment 140 may include a second attachment connector 146, a second enclosure 141, and a second functional unit 148. The second attachment connector 146 may be located at the first attachment end 142 and mechanically compatible with the second base connector 128 of the base handle 120 such that the second attachment connector 146 is coupled to the second base connector 128 via a fast action (e.g., snap action). The second enclosure 141 may be hollow and have a second length which may be the same or different than the first length of the first attachment 130. It may have any suitable shape (e.g., cylinder). The second functional unit 148 may be placed inside the second enclosure 141 and operate by receiving power from the power source 160 via the power connector 125 in the base handle 120.

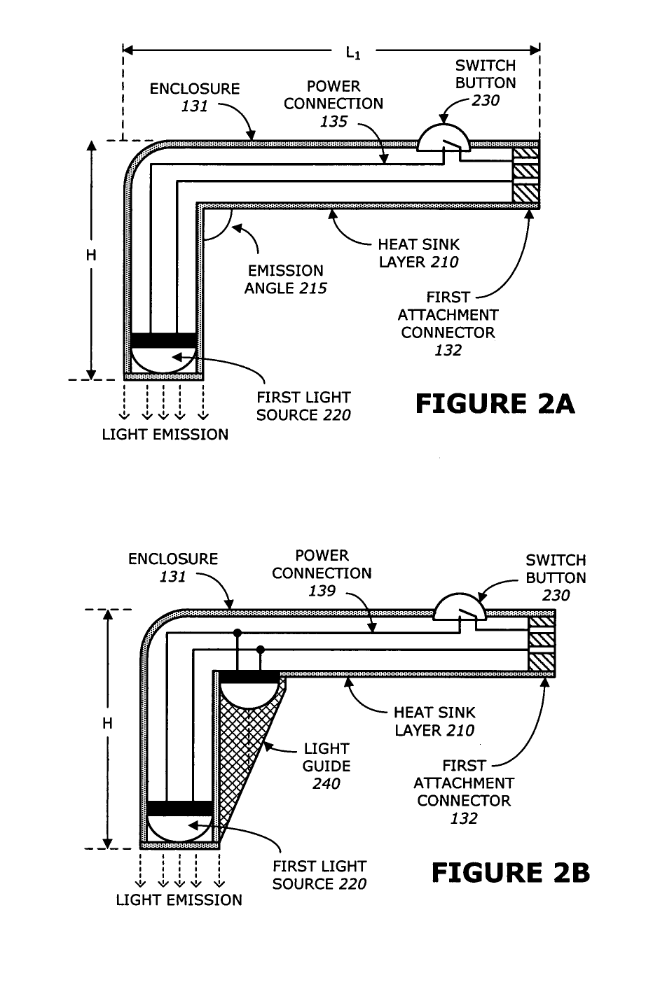

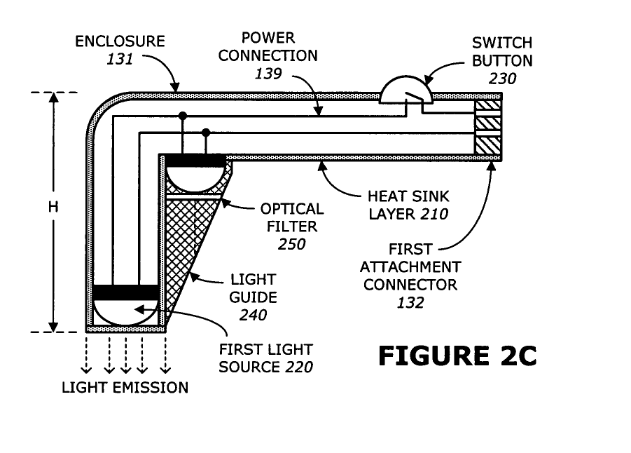

FIG. 2A is a diagram illustrating the first attachment 130 as a curing unit with one light source according to one embodiment of the invention. When the first attachment used as a curing unit together with the instrument tip as a spatula, the combined dental instrument provides a convenient tool for the dentist to perform repetitive intermittent cycles of placement and curing resin composite by just switching the instrument to use the appropriate end.

The first enclosure 131 may be an L-shaped enclosure. It may have a long portion of length L₁ and a short portion of height H. The length L₁ may range from 30 mm to 80 mm. The height H may range from 10 mm to 30 mm. The long portion and the short portion form an emission angle 215. The emission angle 215 may range from 80° to 100°. The enclosure 131 may be made of somewhat flexible material so that the short portion may be bent slightly to adjust the emission angle 215 as necessary to direct the light. The first enclosure 131 may have a heat sink layer 210 that is extended along both sides of the long and short portions. The heat sink layer 210 may include a layer of non thermally conducting material such as epoxy.

The first functional unit 136 may be curing unit having a first light source 220 emitting at a wavelength suitable for curing. The first light source 220 may be a blue LED at a wavelength from 400 nm to 480 nm. The blue LED may have a high power of over 800 mW/cm². The high power of the blue LED facilitates the curing process. The first enclosure 131 may have a heat sink layer 210 to dissipate the heat generated by the light source 220.

FIG. 2B is a diagram illustrating the first attachment 130 as a curing unit with two light sources according to one embodiment of the invention.

In this embodiment, the first functional unit 136 may be the same as in FIG. 2A except that there is a second light source 230 and a light guide 240 located at the corner where the long portion and the short portion meet. The second light source 230 may be a violet LED at a wavelength from 380 nm to 420 nm. The light guide 240 directs the second light source 230 to emit light toward the tip of the short portion so that the two light emissions of the first and second light sources 220 and 230 may be combined, mixed, or fused together.

FIG. 2C is a diagram illustrating the first attachment 130 as a curing unit with two light sources and an optical filter according to one embodiment of the invention.

In this embodiment, the first functional unit 136 may be the same as in FIG. 2B except that the second light source 230 may be a white LED and there is a violet optical filter 250 placed in front of the second light source. The effect is to create a violet light similar to the embodiment shown in FIG. 2B.

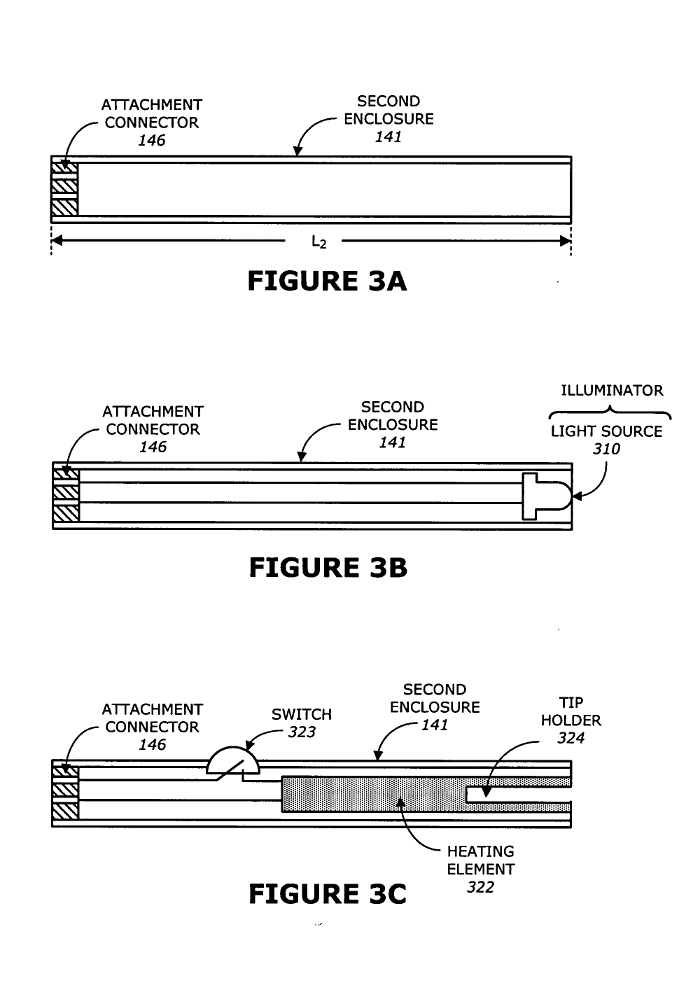

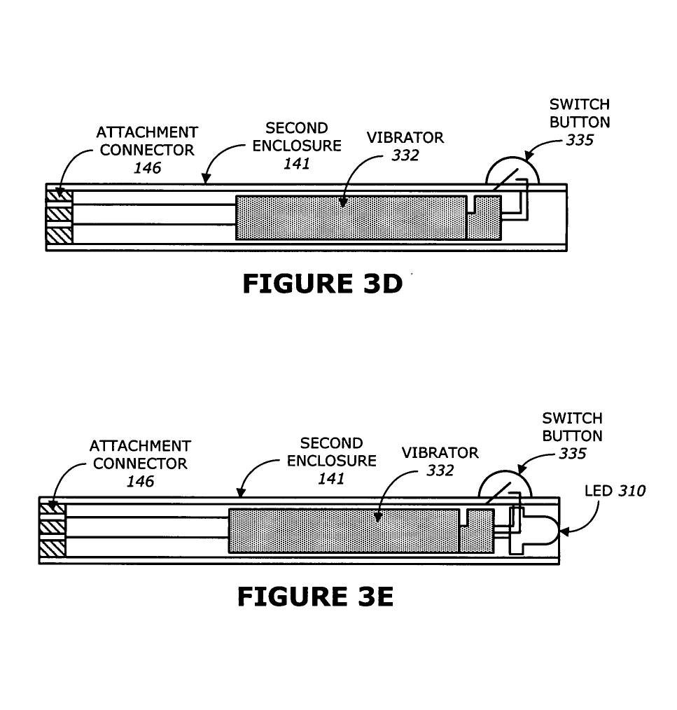

FIG. 3A is a diagram illustrating the second attachment 140 as a hollow enclosure according to one embodiment of the invention.

The second enclosure 141 may be hollow having a length L₂. The length L₂ may range from 30 mm to 80 mm. The second attachment 140 in this embodiment may serve as an extension to the base handle 120.

FIG. 3B is a diagram illustrating the second attachment 140 as an illuminator according to one embodiment of the invention.

In this embodiment, the second functional unit 148 may be an illuminator to emit light to the working area to provide visual comfort to the dentist or the operator using the instrument. It may include a light source 310 extending to the second attachment end 144. The light source 310 may be an LED with sufficient brightness.

FIG. 3C is a diagram illustrating the second attachment 140 as a heating unit according to one embodiment of the invention.

In this embodiment, the second functional unit 148 may be a heating unit to generate heat. It may include a heating element 322 and a switch 323. The heating element 322 may be a heating generating element such as a resistor with low resistance. It may have a tip holder 324 to hold an instrument tip so that the heat may be transferred to the instrument tip. The switch 323 is used to connect or disconnect the electric conduction to the heating element 322 from the power source 160. The switch 323 may be conveniently located on the second enclosure 141 for easy accessibility. The heating unit provides heat to the instrument tip 150 when it is held by the tip holder. This method of heating avoids the inconvenience and potential hazard of using an alcohol lamp or a gas torch to heat the instrument.

FIG. 3D is a diagram illustrating the second attachment 140 as a vibrator unit according to one embodiment of the invention.

In this embodiment, the second functional unit 148 may be a vibrator unit to generate a vibrating or shaking action. It may include a vibrator 332 and a switch 335. The vibrator 332 vibrates when activated. The vibrator 332 may have a motor to control the vibrating movement. The switch 335 is used to connect or disconnect the electric conduction to the vibrator 332 from the power source 160. It may be conveniently located on the second enclosure 141 for easy accessibility. When an instrument tip is attached to the vibrator, the vibrating action caused by the vibrator helps making the material in contact with the instrument tip more flowable, such as composite flowing more easily in deep and narrow spaces, or cement to flow thinly and uniformly when cementing a crown.

FIG. 3E is a diagram illustrating the second attachment 140 as an illuminator and vibrator unit according to one embodiment of the invention.

In this embodiment, the second functional unit 148 includes both the functionalities of an illuminator and a vibrator. It may include the illuminator 310 as in FIG. 3B, the vibrator 332 as in FIG. 3D, and a switch 335. The switch 335 is used to connect or disconnect the electric conduction to the illuminator 310 and the vibrator 332 from the power source 160. It may be conveniently located on the second enclosure 141 for easy accessibility.

Any combination of the above embodiments may be realized. For example, the heating element in FIG. 3C may be combined with the illuminator (FIG. 3B) or the vibrator (FIG. 3D).

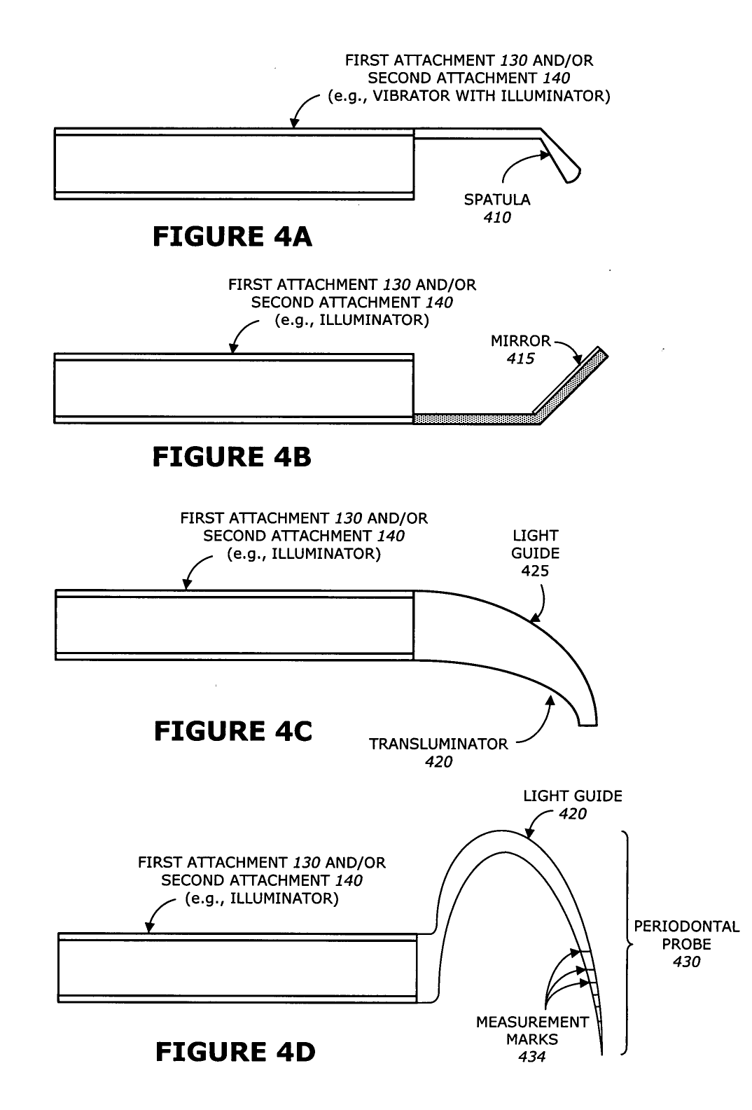

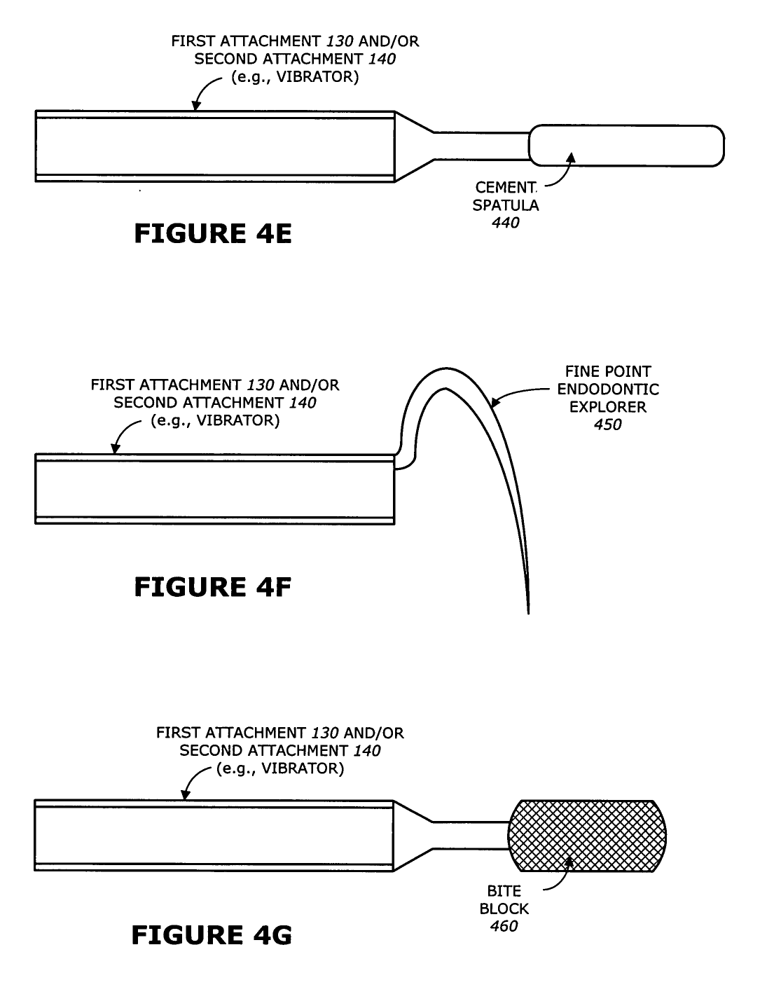

FIG. 4A is a diagram illustrating the instrument tip 150 as a spatula according to one embodiment of the invention. The instrument tip 150 may be attached to the second attachment 140. In this embodiment, the instrument tip 150 may be a spatula 410 attached to the second attachment end 144 of the second attachment 140, or the first attachment 130. The second attachment 140 may be the vibrator with illuminator. The housing of the second attachment 140 may be made of a hard material to maximize the transmission of the vibrations from the vibrator. The spatula 140 is used to place a filling material. It may be attached to the second attachment 140 at the edge of the enclosure.

FIG. 4B is a diagram illustrating the instrument tip 150 as a mirror according to one embodiment of the invention. The instrument tip 150 may be attached to the second attachment 140. In this embodiment, the instrument tip 150 may be a mirror 415 attached to the second attachment end 144 of the second attachment 140. The second attachment 140 may be the illuminator as shown in FIG. 3B. The enclosure of the second attachment 140 may be transparent and have a closed end toward the instrument tip.

FIG. 4C is a diagram illustrating the instrument tip 150 as a transluminator according to one embodiment of the invention. In this embodiment, the instrument tip 150 may be a transluminator 420 attached to the second attachment end 144 of the second attachment 140. The second attachment 140 may be the illuminator as shown in FIG. 3B. The transluminator 420 may have a light guide to direct the light from the second attachment 140 to the tip of the transluminator 420.

FIG. 4D is a diagram illustrating the instrument tip 150 as a periodontal probe according to one embodiment of the invention. In this embodiment, the instrument tip 150 may be a periodontal probe 430 attached to the second attachment end 144 of the second attachment 140. The second attachment 140 may be the illuminator as shown in FIG. 3B. The periodontal probe 430 may include a light guide 432 and measurement marks 434. The light delivered by the illuminator provides visual comfort for the dentist or the operator when measuring the periodontal pockets around the gum of the patient.

FIG. 4E is a diagram illustrating the instrument tip 150 as a cement spatula according to one embodiment of the invention. The instrument tip 150 may be attached to the second attachment 140. In this embodiment, the instrument tip 150 may be a cement spatula 440 attached to the second attachment end 144 of the second attachment 140. The second attachment 140 may be the vibrator as shown in FIG. 3D. The vibration caused by the vibrator allows a fast mixing of cement powder and liquid, leading to a homogeneous result.

FIG. 4F is a diagram illustrating the instrument tip 150 as an endodontic explorer according to one embodiment of the invention. The instrument tip 150 may be attached to the second attachment 140. In this embodiment, the instrument tip 150 may be a fine point endodontic explorer 450 attached to the second attachment end 144 of the second attachment 140. The second attachment 140 may be the vibrator as shown in FIG. 3D. When placing cement or sealer material inside the root canal with the explorer, the vibration caused by the vibrator makes the material more flowable and allows a more complete spread inside the canal than without the vibrator. It may be attached to the second attachment 140 at the edge of the enclosure.

FIG. 4G is a diagram illustrating the instrument tip 150 as a bite bloc according to one embodiment of the invention. The instrument tip 150 may be attached to the second attachment 140. In this embodiment, the instrument tip 150 may be a bite block 460 attached to the second attachment end 144 of the second attachment 140. The bite block 460 may be made of soft plastic. The second attachment 140 may be the vibrator as shown in FIG. 3D. The enclosure of the second attachment 140 may be made of a hard material. When cement a crown, the patient is asked to bite on an object (e.g., a cotton roll, a soft plastic bite block) to exert a pressure on the crown while the cement has not become hard yet. The vibrator may vibrate for a few seconds at the beginning of the pressure. This vibrating action causes the cement to flow more thinly under the crown and allows a better fit at the crown's margins.

FIG. 4H is a diagram illustrating the instrument tip 150 as a gutta percha heater according to one embodiment of the invention. The instrument tip 150 may be attached to the second attachment 140. In this embodiment, the instrument tip 150 may be a gutta percha heater 470 attached to the second attachment end 144 of the second attachment 140. The second attachment may be the heater unit shown in FIG. 3C. The heating element in the heater unit delivers the necessary hear to cut the gutta percha which is the root canal main filling core material.

FIG. 4I is a diagram illustrating the instrument tip 150 as a wax spatula according to one embodiment of the invention. The instrument tip 150 may be attached to the second attachment 140. In this embodiment, the instrument tip 150 may be a wax spatula 480 attached to the second attachment end 144 of the second attachment 140. The second attachment may be the heater unit shown in FIG. 3C. The heating element in the heater unit delivers the necessary hear to work on wax for denture try-in sessions.

FIG. 4J is a diagram illustrating the instrument tip 150 as a heating tip according to one embodiment of the invention. In this embodiment, the instrument tip 150 may be a heating tip 490 attached to the second attachment end 144 of the second attachment 140. The second attachment 140 may include the heating unit as shown in FIG. 3C. The heating tip 490 may be the gutta percha heater or the wax spatula shown in FIGS. 4H and 4I. It may be held at the tip holder 324 of the heating element 322.

A number of configurations to enhance the versatility of the first attachment 130 may be made. The first attachment 130 may include an illuminating unit and a supplementary device having a tip attached to the first enclosure or the illuminating assembly such that the illuminating assembly is positioned at an illuminating distance from the tip.

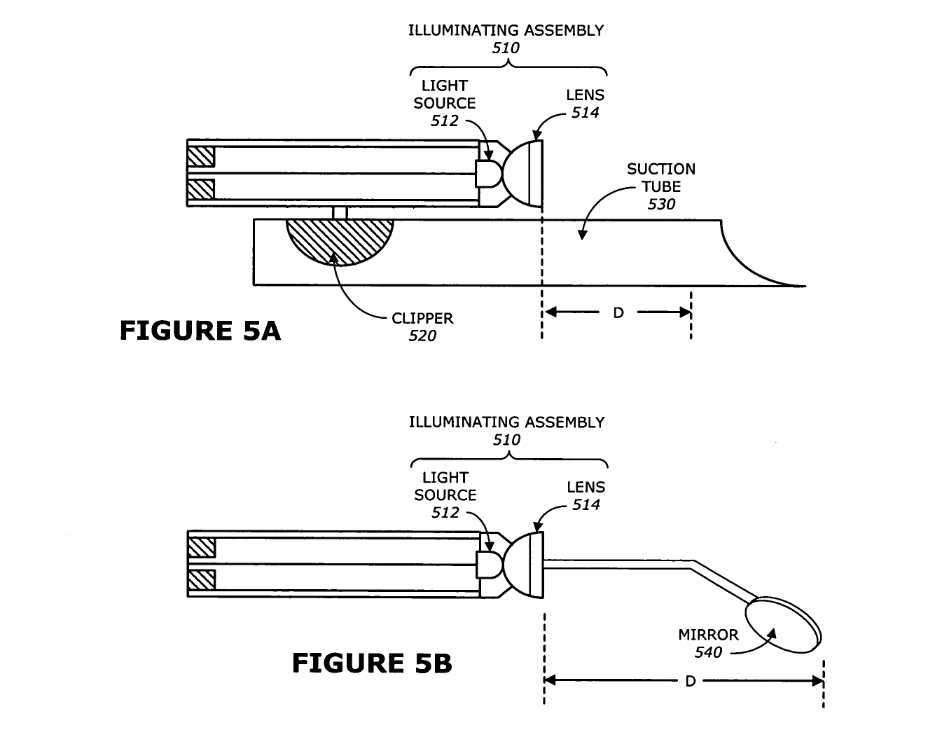

FIG. 5A is a diagram illustrating the first attachment 130 having an illuminating assembly attached to a suction tube according to one embodiment of the invention. The functional unit 136 of the first attachment 130 may be an illuminating assembly 510. The illuminating assembly 510 may include a light source 512 and a lens 514. The light source 512 may be an LED inserted into or attached to the lens 514. The lens 514 may be selected to have a suitable focal length to focus the light to the object being illuminated in the working area. The light intensity therefore is increased. The supplementary device may be a suction tube 530. The suction tube 530 may be firmly attached to the first attachment 130 by a clipper 520. The illuminating distance D between the tip of the suction tube 530 and the illuminating assembly 510 may range from three to eight inches.

FIG. 5B is a diagram illustrating an attachment having an illuminating assembly attached to a mirror according to one embodiment of the invention.

In this embodiment, the illuminating assembly is the similar to the illuminating assembly 510 in FIG. 5A. A mirror 510 may be attached to the center of the illuminating assembly 510. The focused light from the illuminating assembly 510 illuminates directly the area behind the back of the mirror 510 and also reflects the light on the mirror surface. In this manner, the instrument allows illumination in both direct and indirect examinations.

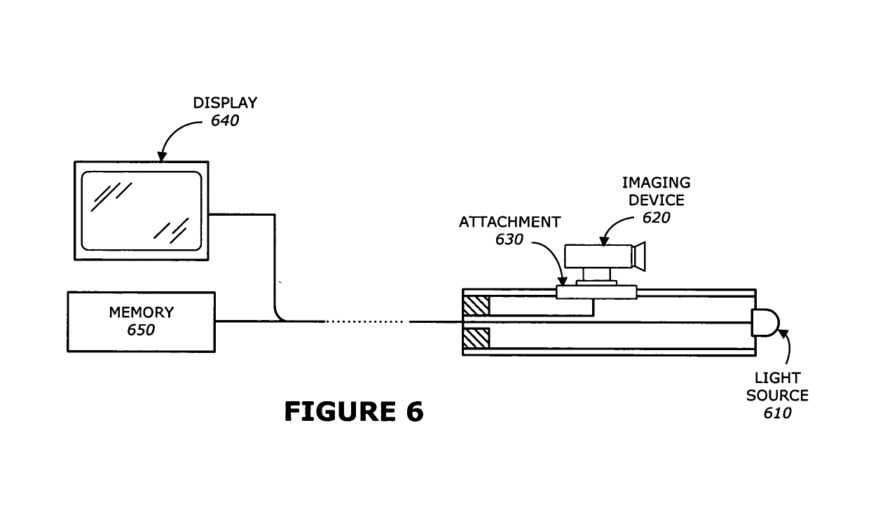

FIG. 6 is a diagram illustrating an attachment having an imaging device according to one embodiment of the invention.

The attachment, either the first attachment 130 or the second attachment 140, has a light source 610 and an imaging device 620. The light source 610 may be an LED with sufficient brightness to provide light for the imaging process. The imaging device 620 is attached to the first enclosure 131 by an attachment mechanism 630 to capture images at the work area. The imaging device 620 acts as an intra-oral camera to capture intra-oral images. The captured images may then be transmitted to a display screen 640 located externally to the first attachment 130. The captured images may also be stored in a memory 650. The imaging device 620 may be a miniature camera or video camera. The display screen may be a small (e.g., 2-inch, 3-inch, or 5-inch) liquid crystal display (LCD) or any other suitable display device. The memory 650 may include a memory card reader or a flash disk device with universal serial bus (USB) interface, such as a high capacity (e.g., 4 GB-16 GB) memory stick. The display screen 640 and the memory 650 may be part of a computer system with processor, memory, input/output devices, etc. Other components such as a frame capture device or imaging processing and analysis software may be employed to analyze or enhance the captured images. The memory 650 may then be transported to transfer to other devices or systems. The display screen 640 may also be used to display the real-time captured images or the stored images. The stored images may be any images including X-ray images and the captured images. The display screen 640 may also be used as a back lit screen for dental X-ray images.

Note that the designation of the first attachment 130 and the second attachment 140 us merely for clarity and convenience. Any one of the first and second attachments 130 and 140 may have the functional unit as described in FIGS. 2A, 2B, 2C, 3A through 3E. Furthermore, the instrument tip 150 may be attached to either the first attachment 130, or the second attachment 140, or both (using two different, or the same, instrument tips). For example, the first attachment 130 may be an illuminator attached to a mirror and the second attachment 140 may be a heater attached to a gutta percha heater.

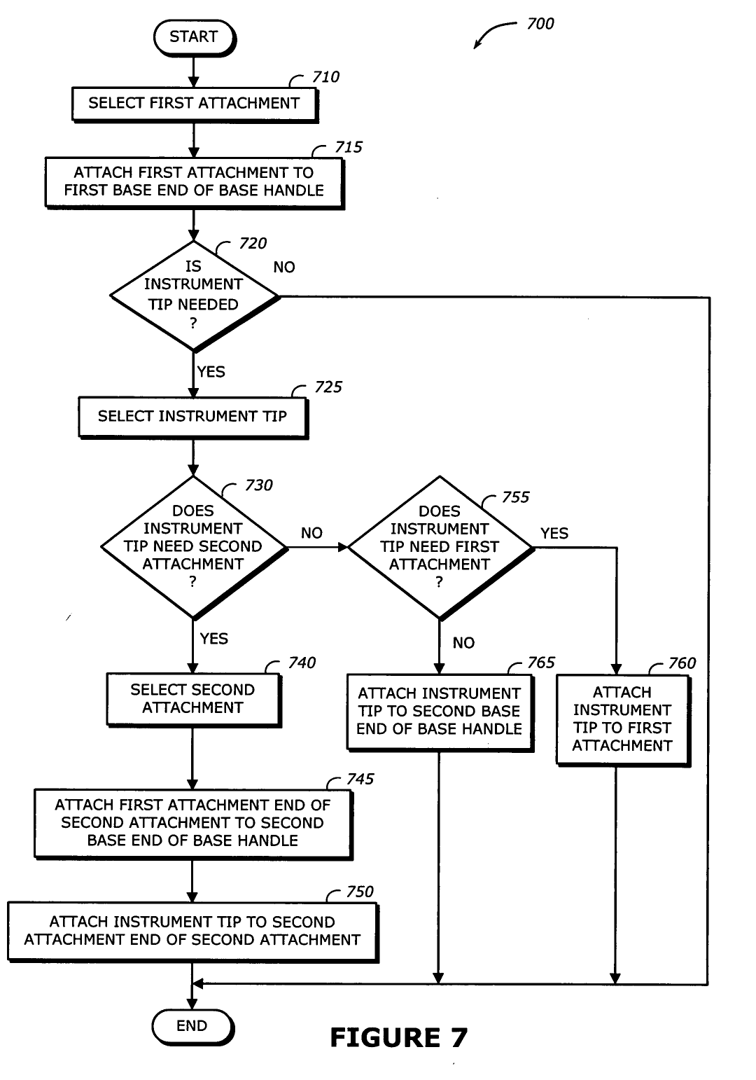

FIG. 7 is a flowchart illustrating a process 700 to use the multi-functional dental instrument according to one embodiment of the invention.

Upon START, the process 700 selects a first attachment from a group of attachments depending on the need (Block 710). For example, the first attachment may be a curing unit, or with a functional unit as an illuminator, a heating unit, a vibrator unit, or a vibrator and illuminator unit, etc.

Then, the process 700 attaches the first attachment to a first base end of a base handle (Block 715). The attachment may be performed using the snap-on connectors at the ends of the first attachment and the base handle.

Next, the process 700 determines if the instrument tip is needed (Block 720). If not, the process 700 is terminated. Otherwise, the process 700 selects the instrument tip to be used with the dental instrument (Block 725). The instrument tip may be selected from tips such as a spatula, a mirror, a transluminator, a periodontal probe, a cement spatula, an endodontic explorer, a crown cementation bite bloc, a gutta percha heater, a wax spatula, and a heating tip.

Then, the process 700 determines if the instrument tip needs a second attachment (Block 730). For example, a periodontal probe may need an illuminator. If not, the process 700 determines if the instrument tip needs the first attachment tip (Block 755). If so, the process 700 attaches the instrument tip to the first attachment (Block 760) and is then terminated. Otherwise, the process 700 attaches the instrument tip directly to a second base end of the base handle (Block 735) and is then terminated. If the instrument tip needs the second attachment, the process 700 selects a second attachment suitable for the instrument tip (Block 740). The process 700 may select a second attachment with a functional unit as an illuminator, a heating unit, a vibrator unit, or a vibrator and illuminator unit, etc.

Then, the process 700 attaches a first attachment end of the selected second attachment to a second base end of the base handle (Block 745). The attachment may be performed using the snap-on connectors at the ends of the second attachment and the base handle. Next, the process 700 attaches the selected instrument tip to a second attachment end of the selected second attachment (Block 750). The process 700 is then terminated.

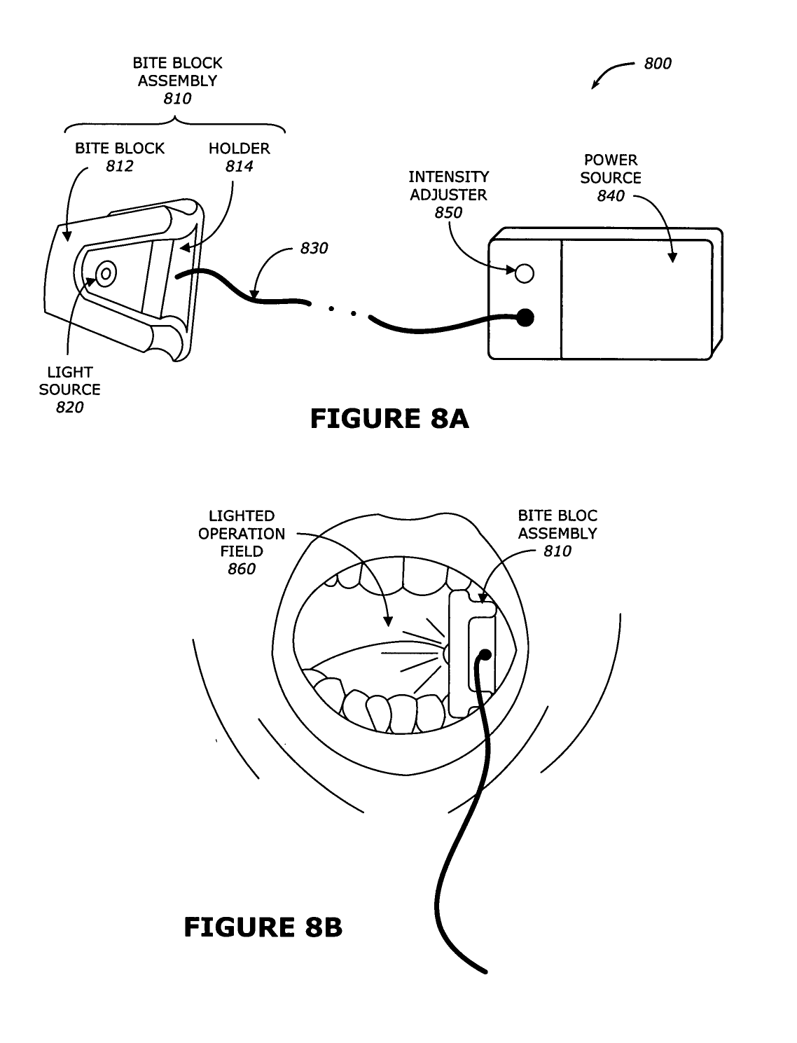

FIG. 8A is a diagram illustrating a bite block unit 800 according to one embodiment of the invention. The bite block unit 800 includes a bite block assembly 810, a light source 820, and a power source 840.

The bite block assembly 810 may be put between the patient's jaws while the patient bites on it. The bite block assembly 810 may be typically put on the opposite side of the operating field. For example, if the dentist works on the patient's left side, the bite block assembly 810 is placed on the patient's right side). The bite block assembly 810 is used to keep the patient's mouth open during the entire operation so that the patient may relax his or her jaw muscles. The bite block assembly 810 includes a bite block 812 and a holder 814. The bite block 812 may be any suitable bite block used in dental work It may be made of relatively soft plastic. The holder 814 provides a mechanical support for the bit block 812. It is embedded in the bite block 812. It may be made of heat-dissipating material and serve as a heat sink for the light source 820. The light source 820 may be a white high intensity LED. It directs the light toward the operating field when the bite block assembly 810 is put in place inside the patient's mouth. The light source 820 may be attached firmly to the holder 814. The holder 814 also provides electrical connections to connect the light source 820 to the power source 840.

The light source 820 obtains power from the power source 840 through a connection wire 830. The power source 840 may be similar to the power source 160 shown in FIG. 1. The power source 840 may have an intensity adjuster 850 to adjust the intensity of the light source 820 by varying the amount of power or current from the power source 830.

FIG. 8B is a diagram illustrating the bite block assembly 810 used in a patient's mouth according to one embodiment of the invention.

The bite block assembly 810 is placed on one side of the patient mouth. The light source 820 directs the light toward the operating field 860 which is on the opposite side of the bite block assembly 810. The bite block assembly 810 therefore serves two purposes: one is to serve as a conventional bite block, and one is to provide illumination inside the patient's mouth.

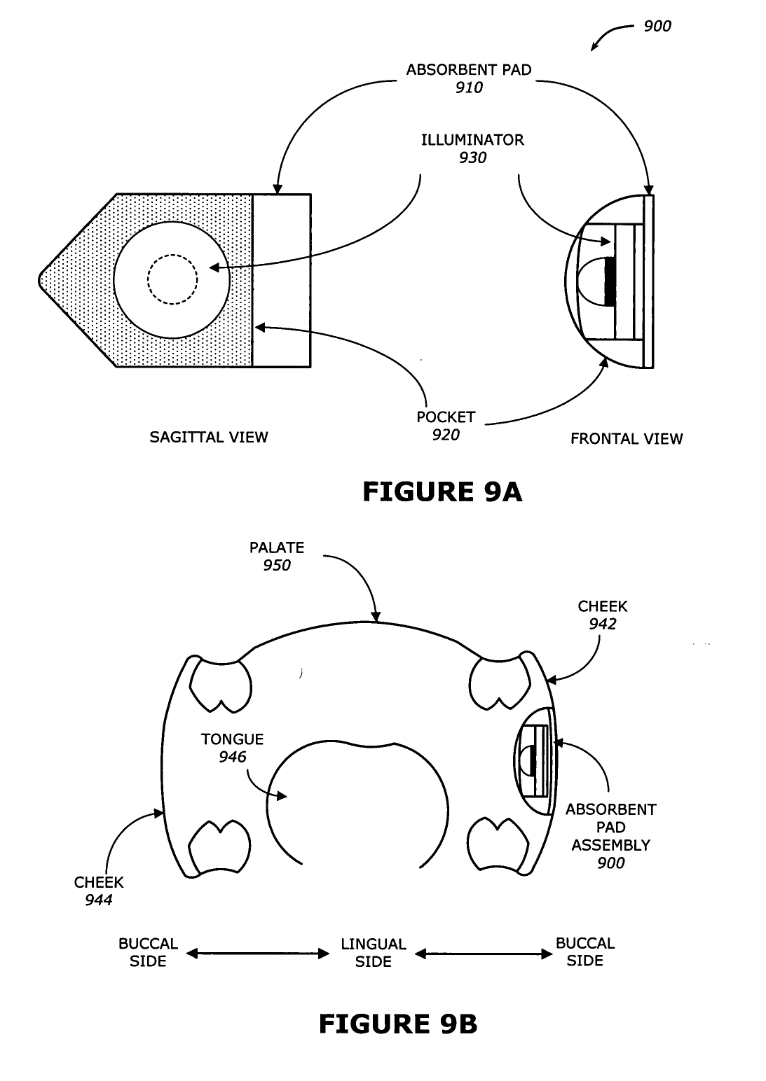

FIG. 9A is a diagram illustrating an illuminator and absorbent pad assembly 900 according to one embodiment of the invention. The illuminator and absorbent pad assembly 900 includes an absorbent pad 910, and illuminator 930, and a pocket 920. The absorbent pad 910 is a saliva absorbent pad. The illuminator 930 may be a light source (e.g., an LED) attached to the absorbent pad to generate light when it is powered by a power source. The power source may be batteries or an external power source. The pocket 920 houses the illuminator 930.

FIG. 9B is a diagram illustrating the illuminator and absorbent pad assembly 900 used in a patient's mouth according to one embodiment of the invention. The absorbent pad assembly 900 described in FIG. 9A may be placed in a patient's mouth to provide light to the working area. The patient's mouth may include cheeks 942 and 944, tongue 946, and palate 950. The illuminator and absorbent pad assembly 900 may be placed in any convenient place. For example, it may be placed on a cheek 942 opposite of cheek 944.

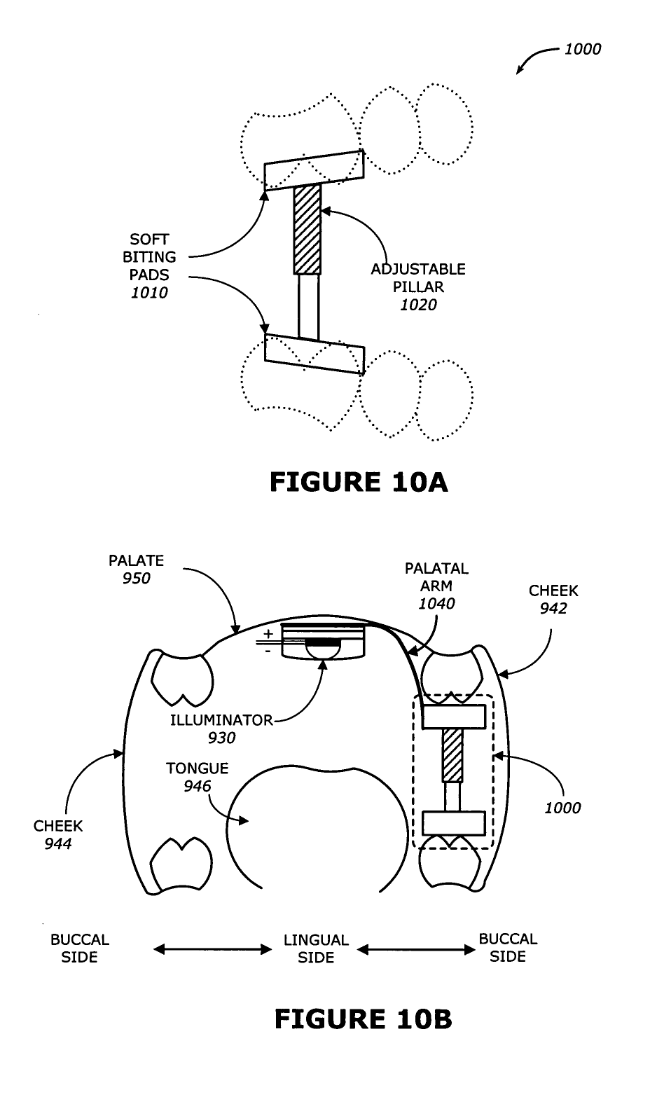

FIG. 10A is a diagram illustrating an adjustable bite block assembly 1000 according to one embodiment of the invention. The adjustable bite block assembly 1000 is shown in a sagittal view. It includes soft biting pads 1010 and adjustable pillar 1020. The adjustable pillar 1020 is positioned between the two soft biting pads 1010. Its length may be adjusted accordingly to create a pressure on the upper and lower teeth when used in a patient's mouth.

FIG. 10B is a diagram illustrating the adjustable bite block assembly 1000 used in a patient's mouth according to one embodiment of the invention. The adjustable bite block assembly 1000 may be used together with the illuminator 930 shown in FIG. 9A. For example, the adjustable bite block assembly 1000 may be placed between the upper and lower teeth on the cheek 942 and the illuminator 930 may be placed on the palate 950. A palate arm 1040 connecting the adjustable bite block assembly 1000 with the illuminator 930.

While the invention has been described in terms of several embodiments, those of ordinary skill in the art will recognize that the invention is not limited to the embodiments described, but can be practiced with modification and alteration within the spirit and scope of the appended claims. The description is thus to be regarded as illustrative instead of limiting.

What is claimed is:

1. An apparatus comprising:
a base handle having first and second base ends opposite to each other; the base handle comprising a power connector to connect to a power source and a first base connector at the first base end; and
a first attachment coupled to the first base connector at the first base end, the first attachment being powered by the power source to perform a first dental task at a working area.

2. The apparatus of claim 1 further comprising:
an instrument tip coupled to the base handle at the second base end to perform a second dental task different than the first dental task.

3. The apparatus of claim 2 wherein the base handle further comprises:
a second base connector at the second base end.

4. The apparatus of claim 3 further comprising:
a second attachment having first and second attachment ends opposite to each other, the first attachment end being coupled to the second base connector at the second base end, the second attachment end being attached to the instrument tip to operate the instrument tip.

5. The apparatus of claim 1 wherein base handle comprises:
a housing to house the power connector and the first base connector, the first base connector having connection to the power connector to receive power from the power source.

6. The apparatus of claim 1 wherein the power source is connected to the power connector inside the housing or outside the housing via a connection wire.

7. The apparatus of claim 1 wherein the first attachment comprises:
a first attachment connector mechanically compatible with the first base connector such that the first attachment is coupled to the first base connector via a fast action.

8. The apparatus of claim 7 wherein the first attachment further comprises:
a first enclosure having a first length.

9. The apparatus of claim 8 wherein the first attachment further comprises:
a first functional unit placed inside the first enclosure and operating by receiving power from the power source via the power connector.

10. The apparatus of claim 9 wherein the first functional device is a curing unit having a light source emitting at a wavelength suitable for curing.

11. The apparatus of claim 4 wherein the second attachment comprises:
a second attachment connector located at the first attachment end and mechanically compatible with the second base connector such that the second attachment connector is coupled to the second base connector via a fast action.

12. The apparatus of claim 11 wherein the second attachment comprises:
a second enclosure having a second length.

13. The apparatus of claim 12 wherein the second attachment further comprises:
a second functional unit placed inside the second enclosure and operating by receiving power from the power source via the power connector.

14. The apparatus of claim 13 wherein the second functional unit is one of an illuminator having a light source to illuminate the working area, a heating unit having a heating element with a tip holder, a vibrator unit having a vibrator with a switch button located on the enclosure, a vibrator and illuminator unit including the illuminator and the vibrator unit.

15. The apparatus of claim 14 wherein the instrument tip is one of a spatula, a mirror, a transluminator, a periodontal probe, a cement spatula, an endodontic explorer, a crown cementation bite bloc, a gutta percha heater, a wax spatula, and a heating tip held by the tip holder.

16. The apparatus of claim 9 wherein the first functional unit comprises:
an illuminating assembly having a light source attached to a lens.

17. The apparatus of claim 16 further comprising:
a supplementary device having a tip attached to the first enclosure or the illuminating assembly such that the illuminating assembly is positioned at an illuminating distance from the tip.

18. The apparatus of claim 17 wherein the supplementary device is one of a suction tube and a mirror and the illuminating distance ranges from one inches to five inches.

19. The apparatus of claim 9 wherein the first functional unit comprises:
a light source.

20. The apparatus of claim 19 wherein the first attachment further comprises:
an imaging device attached to the first hollow enclosure to capture images at the work area, the captured images being transmitted to a display screen located externally to the first attachment.

21. A system comprising:
a power source to generate power;
a multi-function dental instrument receiving the power from the power box, the multi-function dental instrument comprising:
a base handle having first and second base ends opposite to each other; the base handle comprising a power connector to connect to the power source and a first base connector at the first base end, and
a first attachment coupled to the first base connector at the first base end, the first attachment being powered by the power source to perform a first dental task at a working area.

22. The system of claim 21 wherein the multi-function dental instrument further comprises:
an instrument tip coupled to the base handle at the second base end to perform a second dental task different than the first dental task.

23. The system of claim 22 wherein the base handle further comprises:
a second base connector at the second base end.

24. The system of claim 23 further comprising:
a second attachment having first and second attachment ends opposite to each other, the first attachment end being coupled to the second base connector at the second base end, the second attachment end being attached to the instrument tip to operate the instrument tip.

25. The system of claim 21 wherein the first attachment comprises:
a first attachment connector mechanically compatible with the first base connector such that the first attachment is coupled to the first base connectors via a fast action.

26. The system of claim 25 wherein the first attachment comprises:
a first hollow enclosure having a first length.

27. The system of claim 26 wherein the first attachment further comprises:
a first functional device placed inside the first hollow enclosure and operating by receiving power from the power source via the power connector.

28. The system of claim 27 wherein the first functional unit comprises:
a light source.

29. The system of claim 28 wherein the first attachment further comprises:
an imaging device attached to the first hollow enclosure to capture images at the work area, the captured images being transmitted to a display screen located externally to the first attachment.

30. The system of claim 29 further comprising:
a memory unit to store the captured images.

31. An apparatus comprising:
a bite block;
a holder embedded in the bite block; and
a light source attached to the holder to illuminate an operation field, the light source being powered by a power source.

32. The apparatus of claim 31 wherein the holder is made of a heat dissipating material.

33. The apparatus of claim 31 wherein the light source is a white light emitting diode (LED).

34. The apparatus of claim 33 wherein the light source has an intensity adjusted by an intensity adjuster.

35. The apparatus of claim 31 wherein the bite block is made of soft plastic.

36. An apparatus comprising:
an absorbent pad; and
an illuminator having a light source attached to the absorbent pad to generate light when powered by a power source.

37. The apparatus of claim 36 further comprising:
a pocket to house at least one of the absorbent pad and the illuminator.

38. The apparatus of claim 36 wherein the light source is a white light emitting diode (LED).

39. An apparatus comprising:
first and second biting pads; and
a pillar between the first and second biting pads having a length adjusted according to distance between upper and lower teeth.

40. The apparatus of claim 39 further comprising:
an illuminator having a light source attached to one of the first and second biting pads by a palatal arm.

41. The apparatus of claim 40 wherein the light source is a white light emitting diode (LED).
